(19) United States
(12) Patent Application Publication
(10) Pub. No.: US 2017/0065691 A1
(43) Pub. Date: Mar. 9, 2017

(54) BRAIN CANCER IMMUNOTHERAPY

(71) Applicant: University of Southern California, Los Angeles, CA (US)

(72) Inventors: Thomas C. Chen, La Cañada Flintridge, CA (US); Florence M. Hofman, Venice, CA (US)

(73) Assignee: University of Southern California, Los Angeles, CA (US)

(21) Appl. No.: 15/122,759

(22) PCT Filed: Mar. 18, 2015
(86) PCT No.: PCT/US15/21317

§ 371 Date: Aug. 31, 2016

Related U.S. Application Data
(60) Provisional application No. 61/968,186, filed on Mar. 20, 2014.

(51) Int. Cl. A61K 39/00; A61K 9/00

(52) U.S. Cl. CPC A61K 39/0011; A61K 9/0085; A61K 2039/5154; A61K 2039/54; A61K 2039/545

(57) ABSTRACT

The invention describes immunotherapies for treating various cancers in nervous system, particularly brain cancer. In various embodiments, the method may comprise: obtaining a tumor tissue from the subject; preparing a tumor cell lysate from the tumor tissue; isolating an immune cell from the subject; priming the immune cell against the tumor cell lysate. In various embodiments, intraventricular delivery of dendritic cells for brain cancer immunotherapy is disclosed.

### FIELD OF THE INVENTION

The invention relates to methods, compositions and kits for treating various cancers including but not limited to brain cancer.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Current immunotherapy for brain tumors are focused on the following principles. 1) Use of systemic dendritic cells primed against: a) tumor cell lysates for recurrent malignant gliomas; b) tumor cell antigens and cytokines for universal cocktail; c) stem cell lysates. 2) Use of antibody vaccine directed against specific antigen (e.g., mutated epidermal growth factor receptor; 3) Use of patient tumor cell lysates mixed with allogeneic lysates. 4) Use of primed or engineered T-cells. All of these methodologies are given systemically; and all are well tolerated in human patients, with some evidence of efficacy.

There are two physiological barriers for immune therapy delivery to the brain: blood brain barrier (BBB) and the blood cerebrospinal fluid barrier (blood-CSF barrier). The BBB is formed by the tight junction of cerebral endothelial cells and astrocyte foot plates. The blood-CSF barrier is formed by the tight junctions of choroid plexus epithelial cells called Kolmer cells. Dendritic cells (DC) are the most efficient and effective antigen presenting cells, not usually present in the in the central nervous system (CNS). Mature DC can migrate across the BBB and the blood-CSF barriers. DC can present antigens to T-cells, which are rarely present in the normal brain. However, brain tumors particularly glioblastoma multiforme (GBM) demonstrate high numbers of T lymphocytes, particularly CD8 positive cells. Primed DC are able to activate the T cells found locally, at and around the brain tumor. DC-activated T cells can become cytotoxic T lymphocytes and eventually destroy the tumor. Current dendritic cell therapy for brain tumors consists of harvesting autologous dendritic cells from the patient's blood, exposing the DC to patient's tumor cell lysate or antigens, and then injecting DC subcutaneously back into the patient, with the hope that the DC home to the brain tumor thereby eliciting a more vigorous immune response. This therapy has been shown to have some efficacy; but the immune response generated is still not sufficient to cure a brain cancer or prevent progression over time. Part of this difficulty may be due to the use of systemic delivery of dendritic cells to target a brain cancer, and unintentional sequestration of DC in the liver or spleen. Accordingly, there remains a need in the art for treatment of brain tumors.

### SUMMARY OF THE INVENTION

In this invention, we provide a novel method of delivering dendritic cells intraventricularly for brain cancer immunotherapy.

Various embodiments of the present invention provide a method of treating, preventing, reducing the likelihood of having, reducing the severity of and/or slowing the progression of a nervous system tumor in a subject. The method may consist of or may comprise: providing an immune cell; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system. In various embodiments, the immune cell is primed against a tumor cell lysate, tumor cell antigen, tumor cell cytokine, and/or stem cell lysate.

Various embodiments of the present invention provide a method of treating, preventing, reducing the likelihood of having, reducing the severity of and/or slowing the progression of a nervous system tumor in a subject. The method may consist of or may comprise: providing an immune cell; providing a tumor cell lysate, tumor cell antigen, tumor cell cytokine, and/or stem cell lysate; priming the immune cell against the tumor cell lysate, tumor cell antigen, tumor cell cytokine, and/or stem cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system.

Various embodiments of the present invention provide a method of treating, preventing, reducing the likelihood of having, reducing the severity of and/or slowing the progression of a nervous system tumor in a subject. The method may consist of or may comprise: isolating an immune cell from the subject; priming the immune cell against a tumor cell lysate, tumor cell antigen, tumor cell cytokine, and/or stem cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system. In various embodiments, the tumor cell lysate, tumor cell antigen, tumor cell cytokine, and/or stem cell lysate is prepared from a biological sample. In one embodiment, the biological sample comprises tumor cells, cancerous cells, cells from a tumor, tumor tissue, cancerous tissue, and/or a tumor biopsy.

Various embodiments of the present invention provide a method of treating, preventing, reducing the likelihood of having, reducing the severity of and/or slowing the progression of a nervous system tumor in a subject. The method may consist of or may comprise: obtaining a tumor tissue from the subject; preparing a tumor cell lysate from the tumor tissue; providing an immune cell; priming the immune cell against the tumor cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system.

Various embodiments of the present invention provide a method of treating, preventing, reducing the likelihood of having, reducing the severity of and/or slowing the progression of a nervous system tumor in a subject. The method may consist of or may comprise: obtaining a tumor tissue from the subject; preparing a tumor cell lysate from the tumor tissue; isolating an immune cell from the subject; priming the immune cell against the tumor cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system.

In various embodiments, the subject may be a human. In some embodiments, the subject has been, is being, or will be treated with tumor removal surgery, chemotherapy, and/or radiation therapy.

In various embodiments, the nervous system tumor is a tumor in the central nervous system. In certain embodiments, the nervous system tumor may be brain tumor, glioma, recurrent glioma, malignant glioma, glioblastoma, and/or glioblastoma multiforme (GBM).

In various embodiments, the immune cell may be dendritic cell, engineered dendritic cell, T-cell, or engineered T-cell, or a combination thereof. In some embodiments, the immune cell may be isolated from the subject. In some embodiments, the immune cell may be grown in cell cultures. In various embodiments, the immune cell may be primed against a tumor cell lysate, tumor cell antigen, tumor cell cytokine, and/or stem cell lysate. In some embodiments, the tumor cell lysate may comprise or may consist of lysate prepared or derived from the nervous system tumor in the subject. In other embodiments, the tumor cell lysate may comprise or may consist of lysate prepared or derived from a nervous system tumor in another subject other than the subject who has been, is being, or will be treated by a method described herein.

In various embodiments, the immune cell may be administered into a ventricle of the nervous system. In various embodiments, the immune cell is administered via lumbar puncture. In various embodiments, the methods described herein may further comprise placing an Ommaya reservoir into a ventricle of the nervous system and administering a therapeutically effective amount of the immune cell via the Ommaya reservoir.

In various embodiments, the immune cell is administered at about 1×103-1×104, 1×104-1×105, 1×105-1×106, 1×106-1×107, 1×107-1×108, 1×108-1×109, or 1×109-1×1010 cells per dose. In various embodiments, the immune cell is administered once, twice, three or more times. In various embodiments, the immune cell is administered about 1-3 times per day, 1-7 times per week, or 1-30 times per month. In various embodiments, the immune cell is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

Various method described herein may further comprise providing and administering a therapeutically effective amount of a chemotherapeutic agent to the subject. In accordance with the invention, the immune cell and the chemotherapeutic agent are administered concurrently or sequentially.

In various embodiments, the immune cell is provided in a pharmaceutical composition. In some embodiments, the pharmaceutical composition further comprises a pharmaceutically acceptable excipient. In some embodiments, the pharmaceutical composition further comprises a pharmaceutically acceptable carrier. In some embodiments, the pharmaceutical composition may further comprise a chemotherapeutic agent.

Various embodiments of the present invention provide a kit for treating, preventing, reducing the severity of and/or slowing the progression of a nervous system tumor in a subject. The kit may consist of or may consist essentially of or may comprise: a quantify of an immune cell; and instructions for using the immune cell to treat, prevent, reduce the likelihood of having, to reduce the severity of and/or slow the progression of the nervous system tumor in the subject.

Various embodiments of the present invention provide a kit for treating, preventing, reducing the severity of and/or slowing the progression of a nervous system tumor in a subject. The kit may consist of or may consist essentially of or may comprise: a quantify of an immune cell; a quantify of a tumor cell lysate, tumor cell antigen, tumor cell cytokine, and/or stem cell lysate; and instructions for using the immune cell and the tumor cell lysate, tumor cell antigen, tumor cell cytokine, and/or stem cell lysate to treat, prevent, reduce the likelihood of having, reduce the severity of and/or slow the progression of the nervous system tumor in the subject.

Various embodiments of the present invention provide a kit for treating, preventing, reducing the severity of and/or slowing the progression of a nervous system tumor in a subject. The kit may consist of or may consist essentially of or may comprise: a quantify of a tumor cell lysate, tumor cell antigen, tumor cell cytokine, and/or stem cell lysate; and instructions for using the tumor cell lysate, tumor cell antigen, tumor cell cytokine, and/or stem cell lysate to treat, prevent, reduce the likelihood of having, reduce the severity of and/or slow the progression of the nervous system tumor in the subject.

Various kits described herein may further comprise a chemotherapeutic agent, and instructions for using the chemotherapeutic agent to treat, prevent, reduce the likelihood of having, reduce the severity of and/or slow the progression of the nervous system tumor in the subject.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of glioma, delay or slowing of glioma, and amelioration or palliation of symptoms associated with glioma.

“Diseases”, “conditions” and “disease conditions,” as used herein may include, but are in no way limited to any form of malignant neoplastic cell proliferative disorders or diseases. Examples of such disorders include but are not limited to cancer and tumor.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant tumors, as well as dormant tumors or micrometastasis. Cancers which migrate from their original location and seed vital organs can eventually lead to the death of the subject through the functional deterioration of the affected organs. As used herein, the term “invasive” refers to the ability to infiltrate and destroy surrounding tissue. Melanoma is an invasive form of skin tumor. As used herein, the term “carcinoma” refers to a cancer arising from epithelial cells. Examples of cancer include, but are not limited to, nervous system tumor, brain tumor, nerve sheath tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer. Examples of brain tumor include, but are not limited to, benign brain tumor, malignant brain tumor, primary brain tumor, secondary brain tumor, metastatic brain tumor, glioma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor. Nervous system tumor or nervous system neoplasm refers to any tumor affecting the nervous system. A nervous system tumor can be a tumor in the central nervous system (CNS), in the peripheral nervous system (PNS), or in both CNS and PNS. Examples of nervous system tumor include but are not limited to brain tumor, nerve sheath tumor, and optic nerve glioma.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intracranial, intraventricular, intrathecal, epidural, intradural, intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

The term “sample” or “biological sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

As used herein, a “subject” means a human or animal. Usually the animal is a vertebrate such as a primate, rodent, domestic animal or game animal Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., brain tumors) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

No immunotherapy via primed dendritic cells has ever been performed via direct delivery into the ventricle, with goal of penetrating the blood-CSF barrier into the brain tumor. Intraventricular delivery of ex-vivo primed DC by administration directly into the ventricle leads to efficient delivery of activated DC through the choroid plexus. This novel delivery allows for a high concentration of dendritic cells into the glioma, less systemic sequestration of dendritic cells, and longer half-life of dendritic cells in the CSF due to limited sequestration and destruction of DC in the liver and spleen. In addition, insertion of Ommaya reservoir will allow for periodic readministration of dendritic cells. Dendritic cells will be reprimed by periodic withdrawal of blood from the patient; stored tumor cell lysates will be used to restimulate DC.

The use of dendritic cells to increase response to brain tumors is well researched. There are a number of protocols and companies that are using dendritic cells exposed to glioma cells (live), glioma cell lysates, glioma stem cells, or glioma growth factors and antigens. These current dendritic cell protocols for malignant gliomas may be immediately incorporated into the novel treatment methods provided herein by utilizing the intraventricular route for delivery.

Intraventricular delivery of dendritic cells for immunotherapy of brain tumors has several advantages. 1) Dendritic cell technology for isolation and activation of human dendritic cells for brain tumors is well established. 2) Intraventricular delivery of dendritic cells has never been performed before, and now provides a novel method of delivery. 3) This intraventricular delivery of DC is more effective than systemic administration. 4) Intraventricular delivery can be well tolerated, without immune complications. Moreover, one can perform administration of dendritic cells via lumbar puncture with administration into the CSF.

### Treatment Methods

In various embodiments, the present invention provides a method of treating, preventing, reducing the likelihood of having, reducing the severity of and/or slowing the progression of a nervous system tumor in a subject. The method consists of or comprises: providing an immune cell; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system. In various embodiments, the immune cell is primed against a tumor cell lysate, tumor cell antigen, tumor cell cytokine, and/or stem cell lysate.

In various embodiments, the present invention provides a method of treating, preventing, reducing the likelihood of having, reducing the severity of and/or slowing the progression of a nervous system tumor in a subject. The method consists of or comprises: providing an immune cell; providing a tumor cell lysate, tumor cell antigen, tumor cell cytokine, and/or stem cell lysate; priming the immune cell against the tumor cell lysate, tumor cell antigen, tumor cell cytokine, and/or stem cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system.

In various embodiments, the present invention provides a method of treating, preventing, reducing the likelihood of having, reducing the severity of and/or slowing the progression of a nervous system tumor in a subject. The method consists of or comprises: isolating an immune cell from the subject; priming the immune cell against a tumor cell lysate, tumor cell antigen, tumor cell cytokine, and/or stem cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system. In various embodiments, the tumor cell lysate, tumor cell antigen, tumor cell cytokine, and/or stem cell lysate is prepared from a biological sample. In one embodiment, the biological sample comprises tumor cells, cancerous cells, cells from a tumor, tumor tissue, cancerous tissue, and/or a tumor biopsy.

In various embodiments, the present invention provides a method of treating, preventing, reducing the likelihood of having, reducing the severity of and/or slowing the progression of a nervous system tumor in a subject. The method consists of or comprises: obtaining a tumor tissue from the subject; preparing a tumor cell lysate from the tumor tissue; providing an immune cell; priming the immune cell against the tumor cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system.

In various embodiments, the present invention provides a method of treating, preventing, reducing the likelihood of having, reducing the severity of and/or slowing the progression of a nervous system tumor in a subject. The method consists of or comprises: obtaining a tumor tissue from the subject; preparing a tumor cell lysate from the tumor tissue; isolating an immune cell from the subject; priming the immune cell against the tumor cell lysate; and administering a therapeutically effective amount of the immune cell into the nervous system of the subject, thereby treating, preventing, reducing the likelihood of having, reducing the severity of and/or slowing the progression of the nervous system tumor in the subject. In certain embodiments, the nervous system tumor is a tumor in the central nervous system.

In various embodiments, the nervous system tumor is a tumor in the central nervous system. Examples of tumors include, but are not limited to, benign brain tumor, malignant brain tumor, primary brain tumor, secondary brain tumor, metastatic brain tumor, glioma, glioblastoma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor.

In various embodiments, the nervous system tumor is brain tumor, glioma, recurrent glioma, malignant glioma, glioblastoma, and/or glioblastoma multiforme (GBM). In certain embodiments, the nervous system tumor is chemoresistant irradiated brain tumor.

In various embodiments, the subject is a human. In various embodiments, the subject is a mammalian subject including but not limited to human, monkey, ape, dog, cat, cow, horse, goat, pig, rabbit, mouse and rat. In various embodiments, the subject has been, is being, or will be treated with tumor removal surgery, chemotherapy, and/or radiation therapy.

In various embodiments, the immune cell is dendritic cell, engineered dendritic cell, T-cell, or engineered T-cell, or a combination thereof. In some embodiments, the immune cell is isolated from the subject.

In various embodiments, the dendritic cells of the present invention are autologous dendritic cells or allogeneic dendritic cells. In various embodiments, dendritic cells suitable for use in accordance with the present invention are isolated or obtained from any tissue in which such cells are found, or are otherwise cultured and provided. Dendritic cells may be found, for example, but in no way limited to, in the bone marrow, in peripheral blood mononuclear cells (PBMCs) of a mammal or in the spleen of a mammal. Additionally, any suitable media that promote the growth of dendritic cells may be used in accordance with the present invention, and may be readily ascertained by one skilled in the art.

In various embodiments, the immune cell is primed against a tumor cell lysate, tumor cell antigen, tumor cell cytokine, and/or stem cell lysate. In some embodiments, the tumor cell lysate comprises lysate prepared or derived from the nervous system tumor in the subject who has been, is being, or will be treated by a method described herein. In other embodiments, the tumor cell lysate comprises lysate prepared or derived from a nervous system tumor in another subject.

In various embodiments, the tumor cell lysate, tumor cell antigen, or tumor cell cytokine is prepared from a nervous system tumor. In some embodiments, the nervous system tumor is a tumor in the central nervous system. In various embodiments, the nervous system tumor is brain tumor, glioma, recurrent glioma, malignant glioma, glioblastoma, and/or glioblastoma multiforme (GBM). In certain embodiments, the nervous system tumor is chemoresistant irradiated brain tumor. In various embodiments, the tumor cell lysate, tumor cell antigen, tumor cell cytokine, and/or stem cell lysate is prepared from a biological sample. In one embodiment, the biological sample comprises tumor cells, cancerous cells, cells from a tumor, tumor tissue, cancerous tissue, and/or a tumor biopsy. In one embodiment, the biological sample is obtained from the subject who has been, is being, or will be treated by a method described herein. In another embodiment, the biological sample is obtained from another subject.

In various embodiments, the immune cell is administered into a ventricle of the nervous system. In accordance with the invention, the ventricle can be any ventricle in the nervous system. Examples of the ventricle include but are not limited, lateral ventricle, right ventricle, left ventricle, third ventricle, fourth ventricle, cerebral aqueduct, and central canal. In some embodiments, the immune cell is administered via lumbar puncture.

In various embodiments, the methods provided herein can further comprise placing an Ommaya reservoir into a ventricle of the nervous system and administering a therapeutically effective amount of the immune cell via the Ommaya reservoir.

Typical dosages of an effective amount of the immune cell can be as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

In various embodiments, the immune cell is administered once a day (SID/QD), twice a day (BID), three times a day (TID), four times a day (QID), or more, so as to administer an effective amount of the immune cell to the subject, where the effective amount is any one or more of the doses described herein.

In various embodiments, the immune cell is administered at about 1×10³-1×10⁴, 1×10⁴-1×10⁵, 1×10⁵-1×10⁶, 1×10⁶-1×10⁷, 1×10⁷-1×10⁸, 1×10⁸-1×10⁹, or 1×10⁹-1×10¹⁰ cells per dose. In various embodiments, the immune cell is administered once, twice, three or more times. In various embodiments, the immune cell is administered about 1-3 times per day, 1-7 times per week, or 1-30 times per month. In various embodiments, the immune cell is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the immune cell is administered, for example, daily, weekly, biweekly, every fortnight and/or monthly at the aforementioned dosages. In certain embodiments, the immune cell is administered to a human.

In some embodiments, the immune cell is administered at the prevention stage of a tumor (i.e., when the subject has not developed the tumor but is likely to or in the process to develop the tumor). In other embodiments, the immune cell is administered at the treatment stage of a tumor (i.e., when the subject has already developed the tumor).

In various embodiments, the methods described herein are used in conjunction with other cancer therapies including but not limited to chemotherapy and/or radiation therapy.

Various method described herein can further comprise providing and administering a therapeutically effective amount of a chemotherapeutic agent to the subject. In accordance with the invention, the immune cell and the chemotherapeutic agent are administered concurrently or sequentially. Still in accordance with the invention, the immune cell is administered before, during or after administering the chemotherapeutic agent. As a non-limiting example, the immune cell is administered, for example, daily, and the chemotherapeutic agent is administered, for example, daily, weekly, biweekly, every fortnight and/or monthly. As another non-limiting example, the immune cell is administered, for example, daily, weekly, biweekly, every fortnight and/or monthly, and the chemotherapeutic agent is administered, for example, daily. Further, each of the immune cell and the chemotherapeutic agent is administered daily, weekly, biweekly, every fortnight and/or monthly, wherein the immune cell is administered on a day different than the day on which the chemotherapeutic agent is administered at the dosages described herein. In some embodiments, the immune cell and the chemotherapeutic agent are in one composition or separate compositions.

Typical dosages of an effective amount of the chemotherapeutic agent can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the chemotherapeutic agent is administered once a day (SID/QD), twice a day (BID), three times a day (TID), four times a day (QID), or more, so as to administer an effective amount of the chemotherapeutic agent to the subject, where the effective amount is any one or more of the doses described herein.

In various embodiments, the chemotherapeutic agent is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, the chemotherapeutic agent is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m², or a combination thereof. In various embodiments, the chemotherapeutic agent is administered once, twice, three or more times. In some embodiments, the chemotherapeutic agent is administered 1-3 times per day, 1-7 times per week, 1-9 times per month, or 1-12 times per year. Still in some embodiments, the chemotherapeutic agent is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the subject, and “mg/m²” refers to mg per m² body surface area of the subject. In certain embodiments, the chemotherapeutic agent is administered to a human.

In various embodiments, the effective amount of the chemotherapeutic agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/kg/day, or a combination thereof. In various embodiments, the effective amount of the chemotherapeutic agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/m²/day, or a combination thereof. In various embodiments, the effective amount of the chemotherapeutic agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg/day, or a combination thereof. In various embodiments, the effective amount of the chemotherapeutic agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m²/day, or a combination thereof. Here, “μg/kg/day” or “mg/kg/day” refers to μg or mg per kg body weight of the subject per day, and “μg/m²/day” or “mg/m²/day” refers to μg or mg per m² body surface area of the subject per day.

In accordance with the invention, the chemotherapeutic agent is administered using the appropriate modes of administration, for instance, the modes of administration recommended by the manufacturer. In accordance with the invention, various routes are utilized to administer the chemotherapeutic agent of the claimed methods, including but not limited to intratumoral, intravenous, intraarterial, intramuscular, subcutaneous, intraperitoneal, aerosol, nasal, via inhalation, oral, transmucosal, transdermal, parenteral, implantable pump or reservoir, continuous infusion, enteral application, topical application, local application, capsules and/or injections. In various embodiments, the chemotherapeutic agent is administered intracranially, intraventricularly, intrathecally, epidurally, intradurally, topically, intravascularly, intravenously, intraarterially, intratumorally, intramuscularly, subcutaneously, intraperitoneally, intranasally, or orally. In various embodiments, the chemotherapeutic agent is administered intraventricularly.

In accordance with the present invention, examples of the chemotherapeutic agent include but are not limited to Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

The method can be adapted or modified to suit its intended purpose. In one embodiment, the method is adapted or modified particularly for the purpose of treating mammalian subjects. In another embodiment, the method is adapted or modified particularly for the purpose of treating human subjects. In further embodiments, the method is adapted or modified for veterinary applications, treating subjects such as, but not limited to, farm animals, domestic animals, and laboratory animals.

### Pharmaceutical Compositions

In various embodies, the immune cell is provided in a pharmaceutical composition. In accordance with various embodiments, the pharmaceutical composition can further comprise a pharmaceutically acceptable excipient. In accordance with various embodiments, the pharmaceutical composition can further comprise a pharmaceutically acceptable carrier.

In various embodiments, the pharmaceutical composition is formulated for intraventricular administration. In various embodiments, the composition is formulated for intracranial, intraventricular, intrathecal, epidural, intradural, topical, intravascular, intravenous, intraarterial, intratumoral, intramuscular, subcutaneous, intraperitoneal, intranasal or oral administration. Preferred pharmaceutical compositions will also exhibit minimal toxicity when administered to a mammal.

In various embodiments, the immune cell in the pharmaceutical composition is provided at about 1×10³-1×10⁴, 1×10⁴-1×10⁵, 1×10⁵-1×10⁶, 1×10⁶-1×10⁷, 1×10⁷-1×10⁸, 1×10⁸-1×10⁹ or 1×10⁹-1×10¹⁰ cells per dose.

In various embodiments, the pharmaceutical composition further comprises a chemotherapeutic agent. In various embodiments, the chemotherapeutic agent in the composition is provided in mg chemotherapeutic agent per kilogram body weight of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg. In various embodiments, the chemotherapeutic agent in the composition is provided in mg chemotherapeutic agent per m² body surface area of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m².

In various embodiments, the pharmaceutical composition is administered 1-3 times per day, 1-7 times per week, or 1-30 times per month. In various embodiments, the pharmaceutical composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In accordance with the invention, the pharmaceutical composition is formulated for intraventricular administration. In various embodiments, the pharmaceutical composition is administered once a day (SID/QD), twice a day (BID), three times a day (TID), four times a day (QID), or more, so as to administer an effective amount of the immune cell to the subject, where the effective amount is any one or more of the doses described herein.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

Polymers formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

The pharmaceutical composition according to the invention can also be a bead system for delivering the therapeutic agent to the target cells. For example, pectin/zein hydrogel bead system may be used to deliver Neuregulin-4 or a pharmaceutical equivalent, analog, derivative or a salt thereof, to the target cells in the subject (Yan F. et al., J Clin Invest. 2011 June; 121(6):2242-53).

### Kits of the Invention

In various embodiments, the present invention provides a kit for treating, preventing, reducing the severity of and/or slowing the progression of a nervous system tumor in a subject. The kit consists of, or consists essentially of, or comprises: a quantify of an immune cell; and instructions for using the immune cell to treat, prevent, reduce the likelihood of having, reduce the severity of and/or slow the progression of the nervous system tumor in the subject.

In various embodiments, the present invention provides a kit for treating, preventing, reducing the severity of and/or slowing the progression of a nervous system tumor in a subject. The kit consists of, or consists essentially of, or comprises: a quantify of an immune cell; a quantify of a tumor cell lysate, tumor cell antigen, tumor cell cytokine, and/or stem cell lysate; and instructions for using the immune cell and the tumor cell lysate, tumor cell antigen, tumor cell cytokine, and/or stem cell lysate to treat, prevent, reduce the likelihood of having, reduce the severity of and/or slow the progression of the nervous system tumor in the subject.

In various embodiments, the present invention provides a kit for treating, preventing, reducing the severity of and/or slowing the progression of a nervous system tumor in a subject. The kit consists of, or consists essentially of, or comprises: a quantify of a tumor cell lysate, tumor cell antigen, tumor cell cytokine, and/or stem cell lysate; and instructions for using the tumor cell lysate, tumor cell antigen, tumor cell cytokine, and/or stem cell lysate to treat, prevent, reduce the likelihood of having, reduce the severity of and/or slow the progression of the nervous system tumor in the subject.

In various embodiments, the kits described herein can further comprise a chemotherapeutic agent, and instructions for using the chemotherapeutic agent to treat, prevent, reduce the likelihood of having, reduce the severity of and/or slow the progression of the nervous system tumor in the subject.

The kit is an assemblage of materials or components, including at least one of the inventive compositions. Thus, in some embodiments the kit contains a component including a drug delivery molecule complexed with a therapeutic agent, as described above.

The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, spray bottles or cans, diluents, buffers, pharmaceutically acceptable carriers, syringes, catheters, applicators (for example, applicators of cream, gel or lotion etc.), pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the compositions can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in assays and therapies. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### EXAMPLES

The following examples are offered for illustrative purposes only, and are not intended to limit the scope of the present invention in any way.

### Example 1

In-vivo laboratory study using an immunocompetent rat glioma model is performed with intraventricular dendritic cell therapy. 1) rat dendritic cells are isolated from peripheral blood; 2) rat DCs are primed by treatment with lysates of RG2 glioma cells ex-vivo; 3) luciferase labeled RG2 glioma cells are implanted intracranially into an immunocompetent syngeneic Sprague Dawley rat; 4) short term Alzet pump with primed DC is inserted into rat ventricle. The treatment groups are as follows: a) Alzet pump delivering primed DC into the ventricle; b) primed DC delivered systemically via subcutaneous administration; c) Alzet pump delivering vehicle with no cells. Tumor growth is monitored by imaging and animal survival.

### Example 2

Patients with newly diagnosed or recurrent malignant gliomas have tumor removed at the time of surgery. Ommaya reservoir for intraventricular access is placed into the right frontal horn of ventricle at the time of surgery. The resected tumor tissue is prepared as tumor cell lysates. Peripheral blood from patients is obtained after surgery, and DCs are isolated. DCs are primed by incubating DC with tumor cell lysates. Primed DCs are administered directly into the lateral ventricle via Ommaya reservoir. The tumor is monitored by MRI scan with and without gadolinium to assess changes in tumor size every two months. Patients are monitored for progression free survival and overall survival.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the a) description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

What is claimed is:

1. A method of treating, preventing, reducing the likelihood of having, reducing the severity of and/or slowing the progression of a nervous system tumor in a subject, comprising:
providing an immune cell; and
administering a therapeutically effective amount of the immune cell into the nervous system of the subject, thereby treating, preventing, reducing the likelihood of having, reducing the severity of and/or slowing the progression of the nervous system tumor in the subject.

2. The method of claim 1, wherein the nervous system tumor is a tumor in the central nervous system.

3. The method of claim 1, wherein the nervous system tumor is brain tumor, glioma, recurrent glioma, malignant glioma, glioblastoma, and/or glioblastoma multiforme (GBM).

4. The method of claim 1, wherein the subject is a human.

5. The method of claim 1, wherein the subject has been, is being, or will be treated with tumor removal surgery, chemotherapy, and/or radiation therapy.

6. The method of claim 1, wherein the immune cell is dendritic cell, engineered dendritic cell, T-cell, or engineered T-cell, or a combination thereof.

7. The method of claim 1, wherein the immune cell is isolated from the subject.

8. The method of claim 1, wherein the immune cell is primed against a tumor cell lysate, tumor cell antigen, tumor cell cytokine, and/or stem cell lysate.

9. The method of claim 8, wherein the tumor cell lysate comprises lysate prepared from the nervous system tumor in the subject.

10. The method of claim 1, wherein the immune cell is administered into a ventricle of the nervous system.

11. The method of claim 1, wherein the immune cell is administered via lumbar puncture.

12. The method of claim 1, further comprising placing an Ommaya reservoir into a ventricle of the nervous system and administering a therapeutically effective amount of the immune cell via the Ommaya reservoir.

13. The method of claim 1, wherein the immune cell is administered at about 1×10³-1×10⁴, 1×10⁴-1×10⁵, 1×10⁵-1×10⁶, 1×10⁶-1×10⁷, 1×10⁷-1×10⁸, 1×10⁸-1×10⁹, or 1×10⁹-1×10¹⁰ cells per dose.

14. The method of claim 1, wherein the immune cell is administered once, twice, three or more times.

15. The method of claim 1, wherein the immune cell is administered about 1-3 times per day, 1-7 times per week, or 1-30 times per month.

16. The method of claim 1, wherein the immune cell is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

17. The method of claim 1, wherein the immune cell is provided in a pharmaceutical composition.

18. A method of treating, preventing, reducing the likelihood of having, reducing the severity of and/or slowing the progression of a nervous system tumor in a subject, comprising:
isolating an immune cell from the subject;
priming the immune cell against a tumor cell lysate, tumor cell antigen, tumor cell cytokine, and/or stem cell lysate; and
administering a therapeutically effective amount of the immune cell into the nervous system of the subject, thereby treating, preventing, reducing the likelihood of having, reducing the severity of and/or slowing the progression of the nervous system tumor in the subject.

19. A method of treating, preventing, reducing the likelihood of having, reducing the severity of and/or slowing the progression of a nervous system tumor in a subject, comprising:
obtaining a tumor tissue from the subject;
preparing a tumor cell lysate from the tumor tissue;
isolating an immune cell from the subject;
priming the immune cell against the tumor cell lysate; and
administering a therapeutically effective amount of the immune cell into the nervous system of the subject, thereby treating, preventing, reducing the likelihood of having, reducing the severity of and/or slowing the progression of the nervous system tumor in the subject.
